(19) United States
(12) Patent Application Publication
(10) Pub. No.: US 2025/0089993 A1
(43) Pub. Date: Mar. 20, 2025

(54) MEDICAL SYSTEMS, DEVICES, AND RELATED METHODS FOR VACUUM AND/OR SPONGE THERAPY

(71) Applicant: BOSTON SCIENTIFIC MEDICAL DEVICE LIMITED, Galway (IE)

(72) Inventor: Deepak Kumar SHARMA, Muzaffarnagar (IN)

(73) Assignee: BOSTON SCIENTIFIC MEDICAL DEVICE LIMITED, Galway (IE)

(21) Appl. No.: 18/830,931

(22) Filed: Sep. 11, 2024

Related U.S. Application Data
(60) Provisional application No. 63/582,568, filed on Sep. 14, 2023.

(51) Int. Cl. A61B 1/00; A61F 13/38

(52) U.S. Cl. CPC A61B 1/00094; A61B 1/00098; A61B 1/00137; A61F 13/38

(57) ABSTRACT

A medical device includes a handle, an end cap, a sponge, one or more wires, and one or more actuation elements. The handle includes a main body and a movable body, and the movable body is movable relative to the main body. The end cap includes a stationary portion and an extendable portion, and the stationary portion includes one or more through-holes. At least a portion of the sponge surrounds a portion of the extendable portion of the end cap. The one or more actuation elements are connected to a portion of the extendable portion of the end cap. Movement of the movable body of the handle controls a movement of the wires and the extendable portion of the end cap. Proximal movement of the actuation elements controls another movement of the extendable portion of the end cap to separate the extendable portion of the end cap from the sponge.

### CROSS-REFERENCE TO RELATED APPLICATIONS

This application claims the benefit of priority under 35 U.S.C. § 119 from U.S. Provisional Application No. 63/582,568, filed Sep. 14, 2023, which is incorporated by reference herein in its entirety.

### TECHNICAL FIELD

Various aspects of this disclosure generally relate to medical systems, devices, and related methods for vacuum and/or sponge therapy that may be used to treat a subject. For example, aspects of the disclosure relate to medical systems, devices, and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the tract. Limited treatment options exist for managing such wounds, which have significant morbidity and mortality rates. Options include surgical re-operation and endoscopic placement of a stent or clips. Surgery is relatively invasive and has high morbidity and mortality rates. Endoscopic stent placement is a less invasive option. The placed stent, however, can migrate from the intended location and/or wall off infection at the treatment site, inhibiting drainage. In some aspects, a sponge, absorbent material, and/or vacuum tube may be positioned at the treatment site to help in recovery, but the positioning and deployment of the sponge, absorbent material, and/or vacuum tube may be time consuming or otherwise difficult or dangerous for the subject.

The systems, devices, and methods of the current disclosure may rectify some of the deficiencies described above or address other aspects of the art.

### SUMMARY

Examples of this disclosure relate to, among other things, systems, devices, and methods for performing one or more medical procedures with the medical systems and devices. Each of the examples disclosed herein may include one or more of the features described in connection with any of the other disclosed examples.

In one example, a medical device may include a handle, an end cap, a sponge, one or more wires, and one or more actuation elements. The handle may include a main body and a movable body, and the movable body is movable relative to the main body. The end cap may include a stationary portion and an extendable portion, and the stationary portion may include one or more through-holes. At least a portion of the sponge may surround a portion of the extendable portion of the end cap. The one or more wires may extend from the movable body of the handle, through the stationary portion of the end cap, and to the extendable portion of the end cap. The one or more actuation elements may be connected to a portion of the extendable portion of the end cap. Movement of the movable body of the handle may control a movement of the one or more wires and the extendable portion of the end cap. Proximal movement of the one or more actuation elements may control another movement of the extendable portion of the end cap to separate the extendable portion of the end cap from the sponge.

The medical device may include one or more of the following aspects. The extendable portion of the end cap may include a band, and the sponge may be positioned radially around the band. The one or more wires may be coupled to the band to extend and/or retract the band and the sponge. The main body may include a longitudinally extending slot, and the movable body may be moveable along at least a portion of the longitudinally extending slot. The medical device may further include a vacuum tube. A distal end of the vacuum tube may be coupled to a proximal portion of the sponge. The distal end of the vacuum tube may be embedded within portion of the sponge. A proximal end of the vacuum tube may be coupled to a vacuum or negative pressure source.

The medical device may further include one or more tubes extending from the main body of the handle to the stationary portion of the end cap. The one or more wires may be each movable within a respective one of the one or more tubes. Distal ends of the one or more tubes may be fixed to the stationary portion of the end cap at the through-holes. The one or more through-holes may include two through-holes. The one or more wires may include two wires, and the one or more tubes may include two tubes. The two through-holes may be evenly spaced around the stationary portion of the end cap. The sponge may expand radially inward when separated from the extendable portion of the end cap. The handle may include a ring at a proximal end of the handle. The end cap may be configured to be coupled to an exterior of a distal portion of an insertion shaft of an insertion device. The medical device may further include an actuator. Proximal ends of each of the one or more actuation elements may be coupled to the actuator such that rotation of the actuator proximally retracts the one or more threads to retract the extendable portion and deploy the sponge.

In another aspect, a medical system may include an insertion device, a suction device, and a medical device. The insertion device may include an insertion shaft with a distal end. The suction device may include a suction tube. The medical device may include a handle, and end cap, a sponge, one or more wires, and one or more actuation elements. The handle may include a main body and a movable body, and the movable body may be movable relative to the main body. The end cap may be positioned around a distal portion of the insertion shaft, and the end cap may include an extendable portion. At least a portion of the sponge may surround a portion of the extendable portion of the end cap. The one or more wires may extend from the movable body of the handle, through the one or more through-holes of the stationary portion of the end cap, and to the extendable portion of the end cap. The one or more actuation elements may be connected to a portion of the extendable portion of the end cap. Movement of the movable body of the handle may control a movement of the one or more wires and the extendable portion of the end cap. Proximal movement of the one or more actuation elements may control another movement of the extendable portion of the end cap to separate the extendable portion of the end cap from the sponge.

The medical system may include one or more of the following aspects. The distal end of the insertion device may include a working channel opening, one or more illumination devices, and one or more visualization devices. The medical system may further include an actuator. Proximal ends of each of the one or more actuation elements may be coupled to the actuator such that rotation of the actuator proximally retracts the one or more actuation elements to retract the extendable portion and deploy the sponge.

In yet another aspects, a method of applying suction to a target site includes coupling one or more portions of an insertion device, a medical device, and a suction device to form a medical system. The medical device may include a handle with a movable body, an end cap including a stationary portion and an extendable portion, and a porous body releasably positioned around a portion of the extendable portion. The suction device may include a vacuum tube coupled to the porous body. The method may also include delivering various portions of the medical system to the target site, positioning and/or deploying the porous body, and applying negative pressure or suction to the target site.

The method may further include one or more of the following aspects. Positioning the porous body may include moving the extendable portion of the end cap by moving the movable body of the handle. Deploying the porous body may include proximally retracting one or more actuation elements coupled to extendable portion of the end cap.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 illustrates an exemplary medical system, including an insertion device, a medical device, and a vacuum device, according to aspects of this disclosure.

FIGS. 2A illustrates a perspective view of a distal portion of the medical device, and FIG. 2B illustrates a perspective view of the distal portion of the medical device coupled to a distal portion of the insertion device, according to aspects of the disclosure.

FIG. 3 illustrates a perspective view of the medical device and the insertion device, according to aspects of the disclosure.

FIGS. 4A and 4B illustrate respective perspective views of the distal portion of medical device in an extended configuration relative to the insertion device, according to aspects of this disclosure.

FIG. 5 illustrates the distal portion of the medical device in a released configuration, according to aspects of this disclosure.

FIG. 6 illustrates a proximal portion of the medical device undergoing a manipulation to release the distal portion of the medical device, according to aspects of this disclosure.

FIG. 7 illustrates a proximal portion of another exemplary medical device undergoing a manipulation to release the distal portion of the medical device, according to aspects of this disclosure.

FIGS. 8A and 8B illustrate different views of a sponge portion of the medical device, according to aspects of the disclosure, according to aspects of the disclosure.

FIG. 9 is a flow chart illustrating an exemplary method that may be performed using one or more aspects of this disclosure, according to aspects of this disclosure.

### DETAILED DESCRIPTION

The terms “proximal” and “distal” are used herein to refer to the relative positions of the components of an exemplary medical system and exemplary medical devices. When used herein, “proximal” refers to a position relatively closer to the exterior of the body or closer to a medical professional using the medical system or medical device. In contrast, “distal” refers to a position relatively further away from the medical professional using the medical system or medical device, or closer to the interior of the body. As used herein, the terms “comprises,” “comprising,” “has,” “having,” “includes,” “including,” or other variations thereof, are intended to cover a non-exclusive inclusion, such that a system, device, or method that comprises a list of elements does not include only those elements, but may include other elements not expressly listed or inherent thereto. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “distal” refers to a direction away from a user/toward a treatment site, and the term “proximal” refers to a direction toward a user. Several drawings include arrows labeled “P” and “D,” indicating proximal and distal directions, respectively. Unless stated otherwise, the term “exemplary” is used in the sense of “example” rather than “ideal.” As used herein, the terms “about,” “substantially,” and “approximately,” indicate a range of values within +/−10% of a stated value.

Embodiments of this disclosure include systems, devices, and methods for endoluminal vacuum therapy (EVAC), which is a procedure to treat wounds. For example, EVAC may be used to treat post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a porous body such as, for example, a sponge/porous body (e.g., a vacuum-sealed porous body) at a distal end of a nasogastric tube. The sponge is placed endoscopically into the perforation, leak, or other wound. A proximal end of the nasogastric tube may be connected to a collection container.

In some examples, EVAC includes endoluminal placement of a porous body, for example, the sponge, or other like material into the wound site, including a perforation, cyst, a leak, an anastomosis, etc. Placement of the material may be via a catheter, scope (endoscope, bronchoscope, colonoscope, duodenoscope, gastroscope, etc.), tube, or sheath inserted into the GI tract via a natural orifice. The orifice can be, for example, the nose, mouth, or anus, and the placement can be in any portion of the GI tract, including the esophagus, stomach, duodenum, large intestine, or small intestine. In some examples, the placement may be in or through a wall of the GI tract. Placement of the material can also be in other organs reachable via the GI tract (e.g., the colon). Negative pressure is then applied through the tube or porous body.

In EVAC, negative pressure is delivered to the wound site in the GI tract, for example through a nasogastric tube having a sponge or other absorbent material at its terminal end. The sponge is placed endoscopically into the perforation, leak, or other wound. Negative pressure then is applied. Applying the negative pressure through the sponge in the wound may help accelerate healing, for example, by encouraging local tissue granulation at a wound site and/or help reduce risk of infection. Devices and systems suited for EVAC are limited, however. For example, during EVAC, a sponge may be delivered to a target site using a scope, forceps, and a suture loop at a distal end of the sponge. For example, the forceps may be extended through a working channel of the scope and used to grasp the loop at the distal end of the sponge. The forceps may be used to move and/or position the sponge to a target site as the scope is navigated to the target site, which can present challenges. In some instances, the sponge may block scope visualization as a user moves the sponge through the GI tract and/or to the target site using the forceps. The sponge may also take up space and/or snag on tissue (e.g., in the GI tract) as the scope is maneuvered to the target site. It may difficult to accurately position the sponge relative the target site.

Reference will now be made in detail to examples of this disclosure described above and illustrated in the accompanying drawings. Wherever possible, the same reference numbers will be used throughout the drawings to refer to the same or like parts.

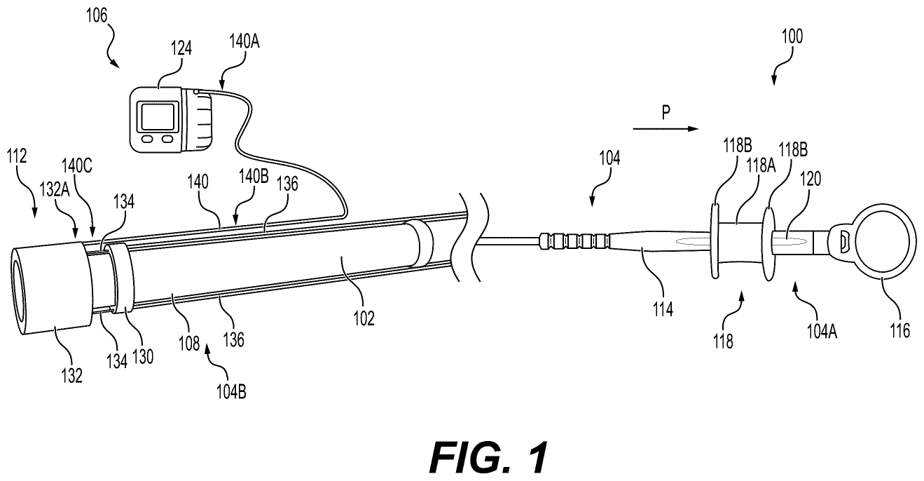

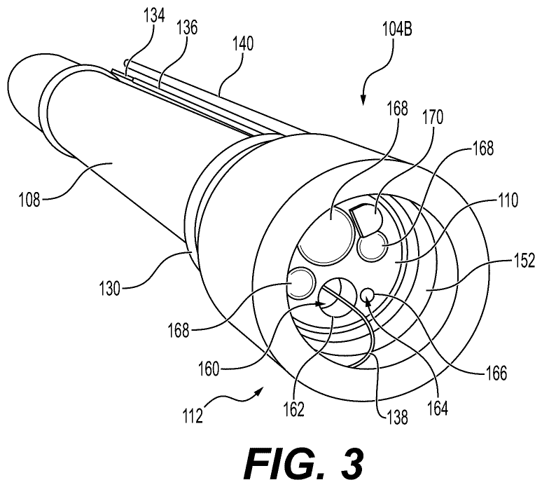

FIG. 1 illustrates an exemplary medical system 100, including, for example, an insertion device 102, a medical device 104, and a suction or vacuum device 106. In some aspects, various portions of system 100 may form and/or be packaged or offered as a kit. Insertion device 102 includes an insertion shaft 108, including a distal end 110 (FIG. 3). Medical device 104 includes a handle 104A and an insertion portion 104B, for example, extending distally from a distal end of handle 104A. As discussed in detail below, insertion portion 104B of medical device 104 includes a distal portion 112, for example, forming an end cap or other coupling element on a distal portion (e.g., distal end 110) of insertion shaft 108. Distal portion 112 includes an adapter 130 and a foam or otherwise porous or absorbent body (e.g., a sponge 132). Adapter 130 may be a stationary portion of distal portion 112, and sponge 132 may be a movable or extendable portion of distal portion 112. Additionally, one or more control elements, for example, cables, wires 134, etc., and one or more coils, tubes, or sheaths 136 may extend from various portions of distal portion 112 to handle 104A. Although not shown in FIG. 1, distal portion 112 may include one or more actuation elements, for example, one or more cables, wires, or threads 138 (FIG. 3).

Adapter 130 may be coupled to a distal portion of insertion shaft 108, and movement of one or more wires 134 (e.g., by one or more manipulations of handle 104A) may control a position of sponge 132 relative to adapter 130, distal end 110 of insertion shaft 108, and a target site. Furthermore, another manipulation of handle 104A may help to deploy sponge 132, for example, via movement or other manipulation of actuation element(s) or thread(s) 138. Vacuum device 106 is coupled to a vacuum tube 140, and vacuum tube 140 may extend from vacuum device 106 to a portion of sponge 132. Additionally, suction or negative pressure may be applied to sponge 132, and thus to the target site to help absorb and/or remove fluid, particulate (e.g., from the target site and/or as sponge 132 degrades), etc. from the target site, which may help to treat the target site (e.g., for one or more endoluminal anastomosis leaks, bleeds, etc.).

Although not shown, one or more portions of medical system 100 may be at least partially enclosed within an outer sheath or outer tube. For example, an outer sheath or outer tube (not shown) may radially surround at least a portion of insertion shaft 108, wires 134, sheaths 136, and/or vacuum tube 140. The outer sheath or outer tube may help deliver or position insertion shaft 108, wires 134, sheaths 136, and/or vacuum tube 140 to the target site, while also allowing relative movement (e.g., longitudinal movement) of the various portions or components of medical system 100.

Insertion device 102 includes insertion shaft 108, which may include a generally cylindrical shaft to be delivered to the target site. Although not shown, insertion device 102 may include a handle with one or more ports, controls, actuators (e.g., to control a deflection or articulation of a distal portion of insertion device 102), electronic components, etc. Insertion device 102 may be an endoscope. Although the disclosure may refer at different points to insertion device 102 being an endoscope, this disclosure is not so limited. For example, unless otherwise specified, duodenoscopes, endoscopes, gastroscopes, endoscopic ultrasonography (“EUS”) scopes, colonoscopes, ureteroscopes, bronchoscopes, laparoscopes, cytoscopes, aspiration scopes, sheaths, catheters, or any other suitable delivery device or insertion device may be used in connection with the systems, devices, elements, assemblies, methods, etc. described herein. Additionally, although the treatment site is discussed herein as being in the subject's GI tract, this disclosure is not so limited, as the treatment site may be any internal lumen, organ, cavity, or other tissue within the subject.

As mentioned, medical device 104 includes handle 104A and insertion portion 104B. Handle 104A includes a main body 114, which may include a ring 116 (e.g., a thumb ring), for example, at a proximal end of main body 114. Handle 104A also includes a first movable member or spool 118. Spool 118 may be an actuator and may include an indented portion 118A (e.g., with a relatively smaller lateral cross-section) and one or more (e.g., two) ridged or extended portions 118B (e.g., with a relatively larger lateral cross-section). In these aspects, indented portion 118A may receive one or more of the user's fingers, such that movement of the user's finger(s) controls the movement of spool 118. Main body 114 may include a slot 120, for example, extending longitudinally through a portion of main body 114, for example, from a position spaced distally from ring 116. A portion of spool 118 may extend into a portion of slot 120, such that spool 118 is movable (e.g., longitudinally movable distally and/or proximally) along slot 120. In these aspects, slot 120 may define a range of movement for spool 118. As discussed in detail below, spool 118 may be movable within slot 120, for example, proximally and/or distally, to control one or more aspects of distal portion 112 of medical device 104. Additionally, although not shown, one or more biasing elements or springs may be positioned within slot 120, for example, to bias distal and/or proximal movement of spool 118.

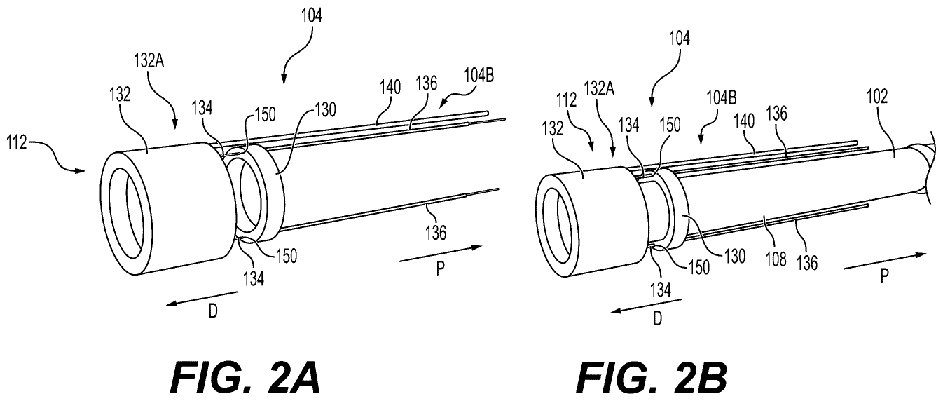

FIGS. 2A and 2B illustrate various details of medical device 104. FIG. 2A illustrates a distal portion of insertion portion 104B of medical device 104 separate from insertion device 102, and FIG. 2B illustrates the distal portion of insertion portion 104B of medical device 104 coupled to insertion shaft 108 of insertion device 102. Insertion portion 104B of medical device 104 includes adapter 130 and sponge 132, for example, in distal portion 112. Additionally, one or more control elements or wires 134, one or more coils, tubes, or sheaths 136, and/or one or more actuation elements or threads 138 may extend from distal portion 112 to handle 104A. Adapter 130 may be coupled to a distal portion of insertion shaft 108, for example, fixedly coupled to a portion of insertion shaft 108. For example, adapter 130 may be coupled to insertion shaft 108 (e.g., at a position proximal of distal end 110, FIG. 3), via a friction fit, an adhesive, a press fit, a crimping, or any other appropriate coupling mechanism. In these aspects, adapter 130 and other components of medical device 104 form an over-tube (over-the-scope) configuration relative to insertion shaft 108 (e.g., a distal portion of insertion shaft 108).

Adapter 130 may be generally ring-shaped, for example, circular to be coupled to an exterior of insertion shaft 108. Adapter 130 includes one or more through-holes 150, for example, extending longitudinally through respective portions of adapter 130. In some aspects, as shown in FIGS. 2A and 2B, adapter 130 may include two through-holes 150. Nevertheless, adapter 130 may include one, three, four, or more through-holes 150. Additionally, through-holes 150 may be evenly spaced (e.g., approximately 180 degrees apart, as shown), or may be unevenly spaced around adapter 130. In any of these aspects, distal ends of coils, tubes, or sheaths 136 may be coupled to, abut, or otherwise terminate at proximal ends or within portions of respective through-holes 150. For example, if there are two through-holes 150, then a distal end of one coil, tube, or sheath 136 may be coupled to, abut, or otherwise terminate at or within one through-hole 150, and a distal end of another coil, tube, or sheath 136 may be coupled to, abut, or otherwise terminate at or within another through-hole 150. Although not shown, proximal ends of coils or sheath 136 may be coupled to, abut, or otherwise terminate at or within a distal portion of handle 104A. In these aspects, coils, tubes, or sheaths 136 may be fixed (i.e., longitudinally fixed) relative to handle 104A and/or insertion shaft 108. Nevertheless, coils, tubes, or sheaths 136 may bend, flex, rotate, or otherwise move with the movement of one or more of handle 104A and/or insertion shaft 108.

As shown in FIGS. 2A and 2B, control elements or wires 134 extend through respective coils, tubes, or sheaths 136. Additionally, each wire 134 extends longitudinally through a respective through-hole 150. Each wire 134 extends distally of through-holes 150 and adapter 130. Additionally, the distal end or distal portion of each wire 134 is coupled to (e.g., directly or indirectly coupled to) a proximal end or a proximal portion 132A of sponge 132. For example, medical device 104 may include or be positioned around one or more bands 152 (FIGS. 3, 4A, 4B, and 8A) rings, caps, support structures, etc., and sponge 132 may be positioned around band 152, for example, in an unexpanded configuration (e.g., FIG. 8A). In some aspects, the distal end or distal portion of each wire 134 may be coupled to a proximal end or proximal portion of band 152. In another aspect, a distal end of each wire 134 may be releasably coupled to a proximal end or proximal portion 132A of sponge 132. Alternatively, the distal end of each wire 134 may be releasably embedded within a portion (e.g., proximal portion 132A) of sponge 132. Furthermore, although not shown, proximal ends of each wire 134 may be coupled to (e.g., directly or indirectly coupled to) spool 118. Accordingly, in any of these aspects, movement of wire(s) 134 (e.g., distally or proximally as controlled by spool 118) may control the position (e.g., extension distally and/or retraction proximally) of sponge 132 relative to distal end 110 and adapter 130.

Sponge 132 may include any suitable biocompatible material that may absorb liquids and/or permit liquid or other materials to pass therethrough via, e.g., negative pressure applied to sponge 132. The material of sponge 132 may be flexible, compressible, porous, hydrophilic, sterile, and/or disposable. Suitable materials include polyurethanes, esters, ethers, composite materials, and/or other medical-grade materials. For example, sponge 132 may be formed of a polyurethane foam. Additionally or alternatively, sponge 132 may include (e.g., be doped with, treated with, infused with, etc.) one or more medications, for example, to assist in promoting tissue growth, preventing infection, assist with pain management, etc.

As discussed below, and as shown in FIG. 3, the movement or positioning of sponge 132 may be visualized via various features of distal end 110. For example, the movement or positioning of sponge 132 may be visualized using one or more illumination devices 168 (e.g., one or more LEDs, optical fibers, and/or other illuminators) and/or one or more visualization devices 170 (e.g., one or more cameras, one or more image sensors, endoscopic viewing elements, optical assemblies including one or more image sensors and one or more lenses, etc.) on distal end 110 of insertion device 102. Furthermore, as discussed below, one or more other manipulations of handle 104A (e.g., of spool 118) may help to release, disconnect, or otherwise deploy sponge 132 from medical device 104.

As shown in FIG. 1, suction or vacuum device 106 includes a vacuum source 124 coupled to a vacuum tube 140, and vacuum tube 140 may extend from vacuum source 124 to a portion of sponge 132. Vacuum tube 140 may include one or more portions. For example, a proximal portion 140A of vacuum tube 140 may be coupled to vacuum source 124, and a distal portion 140B of vacuum tube 140 may extend adjacent to one or more portions of insertion shaft 108 and/or may be coupled to sponge 132. In some aspects, one or more sections of distal portion 140B of vacuum tube 140 may be more flexible than proximal portion 140A of vacuum tube 140, for example, to help allow distal portion 140B to traverse a tortuous path to be delivered to and positioned at the target site.

A distal end 140C of vacuum tube 140 may be coupled to (e.g., directly or indirectly coupled to) a proximal end or a proximal portion of sponge 132. For example, distal end 140C may be coupled to the proximal end or proximal portion 132A of sponge 132 via one or more adhesives, epoxies, etc. Alternatively or additionally, distal end 140C of vacuum tube may be at least partially overmolded or otherwise embedded within a portion (e.g., proximal portion 132A) of sponge 132. In these aspects, vacuum tube 140 (i.e., distal end 140C and distal portion 140B) may move (e.g., extend distally, retract proximally, rotate, deflect, etc.) with the movement of sponge 132. Additionally, suction or negative pressure may be applied to sponge 132, and thus to the target site to help absorb and/or remove fluid, particulates, etc. from the target site, which may help to treat the target site (e.g., for one or more endoluminal anastomosis leaks, bleeds, etc.).

FIG. 3 is a perspective view of a distal portion of system 100, for example, from a distal end looking proximally. Insertion shaft 108 of insertion device 102 may include one or more internal lumens or working channels. For example, insertion shaft 108 may include a working channel 160 terminating distally at a working channel opening 162 at distal end 110. Insertion shaft 108 may also include one or more auxiliary lumens 164, each having a respective auxiliary lumen opening 166. Fluid and/or negative pressure may be delivered or applied to the target site via auxiliary lumen(s) 164. Insertion shaft 108 may have a diameter of approximately 9 mm to approximately 15 mm, for example, approximately 10.5 mm to approximately 12 mm. Working channel 160 may have a diameter of approximately 2 mm to approximately 4mm, for example, approximately 2.8 mm. Furthermore, distal end 110 of insertion shaft 108 may include one or more illumination device(s) 168 (e.g., one or more LEDs, optical fibers, and/or other illuminators). Alternatively or additionally, distal end 110 may include one or more visualization device(s) 170 (e.g., one or more cameras, one or more image sensors, endoscopic viewing elements, optical assemblies including one or more image sensors and one or more lenses, etc.).

Additionally, it is noted that various Figures illustrate distal end 110 of insertion device 102 as being “forward-facing.” In a forward-facing embodiment, working channel opening 162, auxiliary lumen opening 166, illumination device(s) 168, and visualization device(s) 170 face distally (i.e., forward of a distalmost face of distal end 110). In other aspects, however, distal end 110 of insertion device 102 may be “side-facing.” In other words, features of distal end 110 (e.g., working channel opening 162, auxiliary lumen opening 166, illumination device(s) 168, and visualization device(s) 170) may be disposed on a radially outer side of distal end 110, so that they point in a radially outward direction, approximately perpendicularly to a longitudinal axis of distal end 110 of insertion shaft 108. This disclosure also encompasses other configurations of distal end 110 as being “side-facing.” Insertion device 102 may additionally include some components that are forward-facing and other components that are side-facing.

As shown in FIG. 3, one or more actuation elements or threads 138 may extend through working channel 160, for example, and distal to working channel opening 162. Moreover, sponge 132 may be coupled to band 152. For example, sponge 132 may be positioned around an exterior (e.g., a radial exterior) of band 152. Additionally, one or more actuation elements, for example, thread 138, may be coupled to a portion of band 152. For example, a distal end of thread 138 may be coupled (e.g., fixedly coupled to a proximal portion or to a distal portion of band 152. As discussed below, movement or manipulation of thread 138 (e.g., via one or more proximal actuation elements or controls) may help to separate sponge 132 from band 152, for example, to help deploy sponge 132.

Additionally, with sponge 132 and band 152 positioned around an outer portion of insertion shaft 108, sponge 132 and band 152 may not impair or obstruct the visualization of the target site with illumination device(s) 168 and visualization device(s) 170. Furthermore, control elements or wires 134 are movable through through-holes 150 of adapter 130 to position sponge 132 and band 152, and the movement and positioning of sponge 132 and band 152 may be visualized using illumination device(s) 168 and visualization device(s) 170. In some aspects, one or more of sponge 132, band 152, suction tube, wires 134, sheaths 136, or other components or features of distal portion 112 of medical device 106 may include a radiopaque material (e.g., one or more markers, one or more gradients, etc.), or otherwise help the user to visualize sponge 132, band 152, etc.

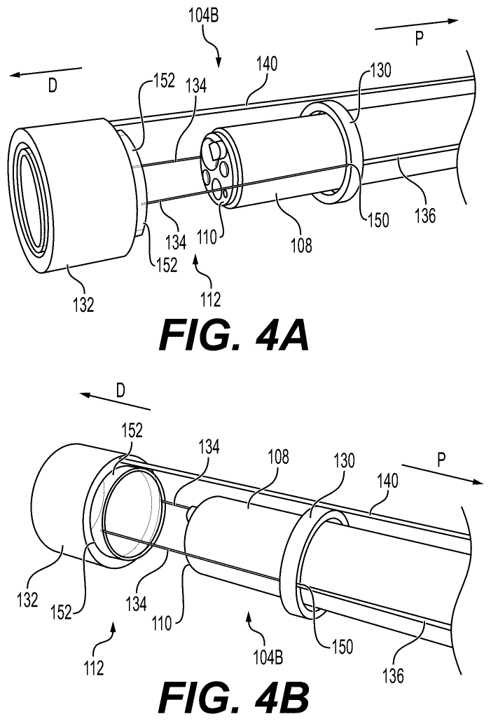

FIGS. 4A and 4B illustrate respective perspective views of sponge 132 and band 152 extended away from distal end 110 of insertion shaft 108 and away from adapter 130, for example, based on movement (i.e., distal extension) of control elements or wires 134. As mentioned above, movement of spool 118 (FIG. 1) distally may extend wires 134 distally. In these aspects, distal movement of spool 118 may extend wire(s) 134 distally, and proximal movement of spool 118 may retract wire(s) 134 proximally. As such, sponge 132 and band 152 may also correspondingly be extended distally and/or retracted proximally, for example, to position sponge 132 relative to insertion shaft 108 and/or the target site. Slot 120 may form a range of movement of spool 118, and thus also form a range of movement for wire(s) 134, sponge 132, and band 152. As mentioned, the movement and/or positioning may be observed, for example, using illumination device(s) 168 and/or visualization device(s) 170, because sponge 132 and/or band 152 may be within a field of view of visualization device(s) 170.

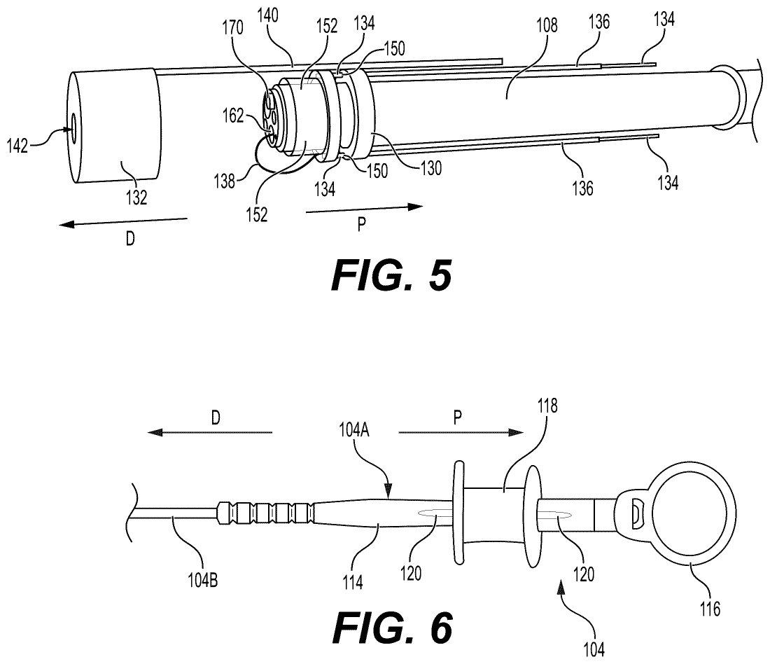

FIG. 5 illustrates a perspective view of sponge 132 deployed or otherwise separated from band 152. As shown, sponge 132 may expand when deployed or separated from band 152. For example, as discussed below, sponge 132 may include a larger thickness (e.g., a radial thickness) or otherwise increase in size when deployed or separated from band 152. For example, sponge 132 may be in a contracted or unexpanded state when positioned around band 152, and separating sponge 132 and band 152 may allow for sponge 132 to transition to an expanded state, as shown in FIG. 5. For example, as discussed above, thread(s) 138 may be coupled to a portion of band 152, and proximal retraction of thread(s) 138 may help to separate sponge 132 from band 152. Additionally or alternatively, as mentioned, proximal portion(s) of band 152 may be coupled to one or more wires 134. As shown, wires 134 may be proximally retracted, urging band 152 proximally, while sponge 132 may remain distally extended. In these aspects, band 152 may be retracted to a position radially outside or otherwise adjacent to or surrounding a distal portion of insertion shaft 108. Additionally, in some aspects, vacuum tube 140 may be used to help deploy and/or position sponge 132. For example, in some aspects, the user may proximally retract wires 134 to proximally retract band 152, while also urging vacuum tube 140 distally, to help deploy sponge 132. Moreover, in some aspects, the user may manipulate vacuum tube 140 (e.g., urge distally, pull proximally, etc.) to help position (e.g., translate) sponge 132 relative to distal end 110 and/or the target site.

FIG. 6 illustrates a side view of handle 104A of medical device 106. As mentioned, handle 104A includes spool 118 that is movable within slot 120. In some aspects, the user(s) may move spool 118 proximally (e.g., toward ring 116) to help deploy sponge 132 and vacuum tube 140 (FIGS. 1-5). For example, moving or urging spool 118 proximally may proximally retract wire(s) 134. In some aspects, moving or urging spool 118 proximally may proximally retract thread 138. Proximal retraction of thread 138 may also proximally retract band 152, for example, pulling band 152 or otherwise separating band 152 from sponge 132.

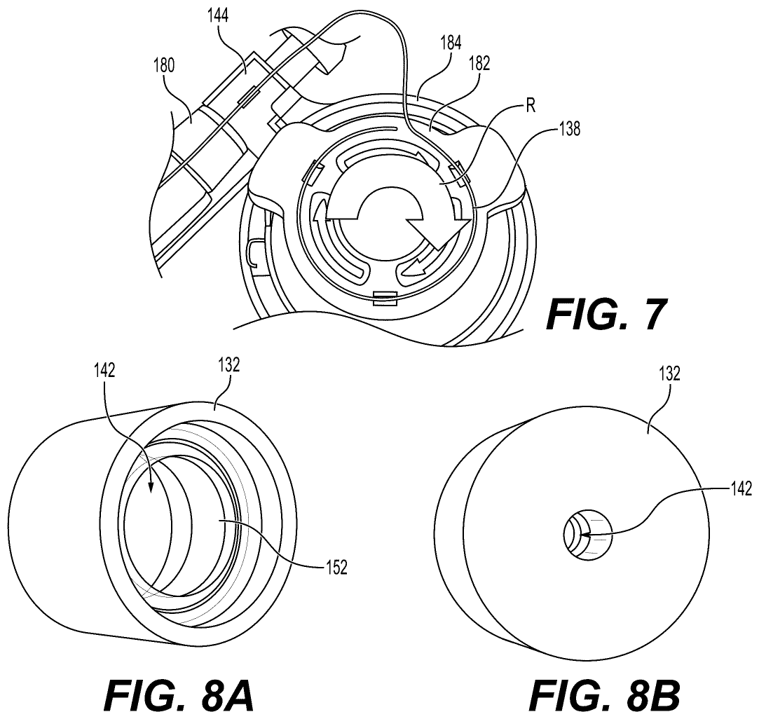

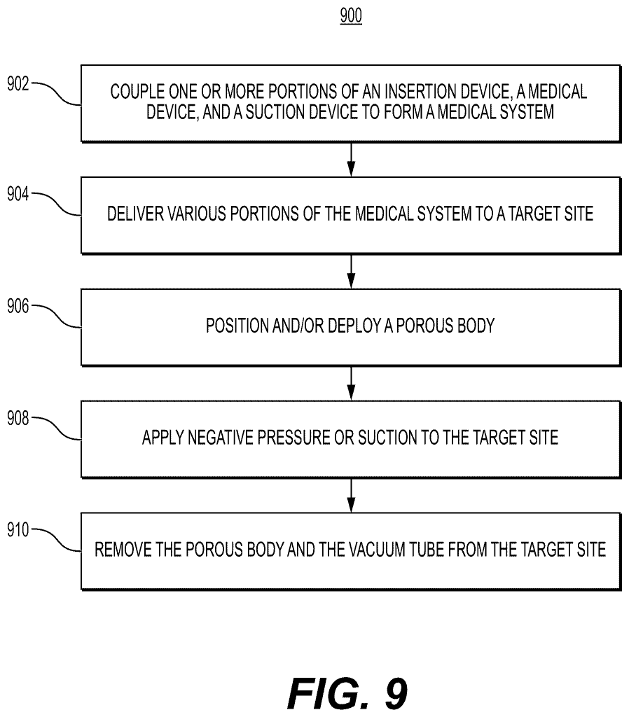

FIG. 7 illustrates a side view of a portion of a handle 180 of another medical device or a portion of medical device that may be included with system 100 (FIG. 1). For example, handle 180 may be a portion of or coupled to a handle of insertion device 102 and/or a portion of or coupled to a portion of handle 104A of medical device 104. For example, handle 180 may be coupled to a port 144 on a portion of the handle of insertion device 102. Handle 180 may include an actuator 182 (e.g., a rotatable knob) and a stationary portion 184 (e.g., a housing). In these aspects, a portion of thread 138 may extend through working channel 160 (FIG. 3) of insertion shaft 108, through a portion of the handle of insertion device 102, and out of port 144 on the handle of insertion device 102. Additionally, a proximal end or portion of thread 138 may be coupled to actuator 182. For example, thread 138 may extend from port 144 and be at least partially wrapped around actuator 182, such that rotation of actuator 182 in a first direction (e.g., in direction R) proximally retracts thread 138. In these aspects, rotation of actuator 182, for example, relative to stationary portion 184 may help to proximally retract thread 138 to separate band 152 from sponge 132 (FIG. 6). In some aspects, actuator 182 may be rotated in direction R to proximally retract thread 138 and separate band 152 and sponge 132.

FIGS. 8A and 8B illustrate various configurations of sponge 132. As mentioned, thread(s) 138 (FIG. 5) may be coupled to a portion of band 152 to control the movement of band 152, for example, relative to sponge 132 to deploy sponge 132. FIG. 8A illustrates sponge 132 in a contracted or unexpanded configuration when surrounding or at least partially positioned around band 152. In some aspects, band 152 may be generally cylindrical or tubular, and may be sized to be positioned around a distal portion of insertion shaft 108 (FIG. 5). Sponge 132 may also be generally cylindrical or tubular, with an inner surface of sponge 132 positioned around the outer surface of band 152 in the contracted or unexpanded configuration. FIG. 8B illustrates sponge 132 in a deployed or expanded configuration, for example, when separated from band 152. As mentioned, band 152 may be proximally retracted relative to sponge 132, for example, via movement of one or more threads 138 (FIG. 5). Once sponge 132 is separated from band 152, sponge 132 may expand. For example, an inner diameter of sponge 132 (e.g., formed by a sponge opening 142) may reduce in size, for example, such that sponge 132 expands radially inward. In some aspects, sponge 132 may expand inward when transitioning from the unexpanded configuration (FIG. 8A) and the expanded configuration (FIG. 8B), reducing the size (e.g., diameter) of sponge opening 142. In these aspects, sponge 132 may maintain an outer diameter or profile in both the unexpanded configuration (FIG. 8A) and the expanded configuration (FIG. 8B). In other aspects, although not shown, sponge 132 may also expand outward, for example, increasing in size radially outward when separated from band 152. In any of these aspects, sponge 132 may be positioned at the target site in the expanded configuration (FIG. 8B) and help absorb and/or remove (e.g., with vacuum tube 140 providing suction or negative pressure) fluid, particulate, etc. from the target site.

Various aspects of this disclosure may be used to perform a method 900, for example, to position and deploy a sponge and vacuum tube at a target site, for example, to help absorb and/or remove fluid, particulate, etc. An initial step 902 includes coupling one or more portions of insertion device 102, medical device 104, and suction device 106, for example, to form medical system 100. For example, step 902 may include coupling adapter 130 to a distal portion of insertion shaft 108 (e.g., proximal of distal end 110). In some aspects, step 902 may include coupling a distal end of vacuum tube 140 to a portion of sponge 132. Alternatively, the distal end of vacuum tube 140 may be fixedly coupled to sponge 132. Moreover, in some aspects, step 902 may include coupling wires 134 to sponge 132 and/or band 152, and/or positioning at least a portion of sponge 132 around band 152 (e.g., transitioning from an expanded configuration to an unexpanded configuration for delivery to the target site). In some aspects, step 902 includes coupling thread 138 to a portion of band 152, and/or feeding thread 138 proximally, for example, through working channel 160 of insertion shaft 108.

Next, a step 904 includes delivering various portions of medical system to a target site, for example, to an endoluminal anastomosis or other cavity. As mentioned, one or more portions of medical system 100 may be delivered to the target site via an outer sheath or outer tube. Additionally, distal portions of medical system 100 may be positioned relative to the target site using one or more visualization techniques (e.g., one or more radiopaque markings on insertion shaft 108, adapter 130, and/or sponge 132) and/or using one or more visualization devices (e.g., illumination device(s) 168 and/or visualization device(s) 170 on distal end 110 of insertion shaft 108). Although not shown, the handle of insertion device 102 may include one or more knobs, levers, etc. to help articulate or otherwise position distal end 110 relative to the target site. Distal portion 112 of medical device 104 may articulate or otherwise be positioned in conjunction with distal end 110.

A step 906 includes positioning and/or deploying porous body (e.g., sponge 132). As mentioned, sponge 132 may be extended and/or retracted relative to adapter 130 and distal end 110 via movement of wire(s) 134 (e.g., as controlled by spool 118). Moreover, the movement and/or positioning of sponge 132 may be visualized, for example, using one or more visualization devices (e.g., illumination device(s) 168 and/or visualization device(s) 170 on distal end 110 of insertion shaft 108). With sponge 132 positioned at a desired location relative to the target site and/or distal end 110, the user may manipulate one or more features of medical device 104 to deploy sponge 132. For example, as mentioned above, the user may proximally retract spool 118 to retract thread(s) 138 and help deploy sponge 132, as discussed with respect to FIG. 6. Alternatively, the user may rotate actuator 182 to retract thread(s) 138 and help deploy sponge 132, as discussed with respect to FIG. 7. In any of these aspects, thread 138 may remain attached to band 152 as sponge 132 separates from band 152. Band 152 may be retracted to fit around a portion of insertion shaft 108, as shown in FIG. 5. Additionally, sponge 132 may transition from the unexpanded state around band 152 (FIG. 8A) to the expanded state separated from band 152 (FIG. 8B). Although not shown or discussed in detail, step 906 may also include one or more other treatments or assessments of the target site.

Next, a step 908 includes applying negative pressure or suction to the target site. Step 908 may include applying negative pressure or suction to the target site via suction device 106 and sponge 132. For example, as discussed above, vacuum tube 140 may be coupled to sponge 132, such that negative pressure or suction from suction device 106 is applied to sponge 132 and the target site. In these aspects, fluid, particulate (e.g., from the target site and/or as sponge 132 degrades), etc. may be absorbed by sponge 132 and/or removed from the target site via suction device 106, and thus removed from the subject via vacuum tube 140. Step 908 may include periodically applying negative pressure or suction to the target site, for example, every hour, every two hours, every six hours, every twelve hours, once a day, once every two days, once every four days, once a week, etc. Alternatively, step 908 may include consistently or constantly applying negative pressure or suction to the target site, for example, for a specific duration, for example, one minute, two minutes, five minutes, ten minutes, 20 minutes, one hour, etc.

Lastly, a step 910 includes removing the porous body (e.g., sponge 132) and the vacuum tube (e.g., vacuum tube 140) from the target site. For example, step 910 may include pulling or urging vacuum tube 140 proximally. Because the distal end of vacuum tube 140 is coupled to a portion of sponge 132, pulling or urging vacuum tube 140 proximally may help remove both vacuum tube 140 and sponge 132 from the target site (e.g., from the subject). In some aspects, the user may repeat one or more steps of method 900, for example, to further treat the target site (or another target site). Step 910 may be performed at a time after step 908 (e.g., after 3-5 days or any other suitable amount of time).

Various aspects of system 100 and/or method 900 may help to treat one or more target sites. For example, insertion shaft 108 may be used to position sponge 132 and vacuum tube 140 at the target site. The user may employ the articulation or movement of insertion shaft 108 to position sponge 132 and vacuum tube 140. The user may also employ one or more visualization techniques (e.g., one or more radiopaque markings on insertion shaft 108, adapter 130, sponge 132) and/or using one or more visualization devices (e.g., illumination device(s) 168 and/or visualization device(s) 170 on distal end 110 of insertion shaft 108 to position sponge 132 and vacuum tube 140. Additionally, handle 104A of medical device 104 (and/or handle 180) may be used to position and/or deploy sponge 132 at the target site, which also allows sponge 132 to expand. Once sponge 132 is positioned, negative pressure or suction may be applied to sponge 132 and/or the target site via vacuum tube 140. For example, proximal portion 140A of vacuum tube 140 may extend to the exterior of the subject, for example, out of an orifice (e.g., mouth, nose, etc.). Accordingly, vacuum source 124 may be activated to apply negative pressure or suction to sponge 132, for example, to help remove excess fluid and/or particulate (e.g., from the target site and/or as sponge 132 degrades) from sponge 132 and/or the target site. In these aspects, applying negative pressure or suction to sponge 132 may help to decrease the size and/or help heal one or more wounds, leaks, etc. at the target site.

Various aspects discussed herein may help to improve the efficacy of treatment and/or recovery from a procedure, for example, a bariatric surgery. Various aspects discussed herein may help to reduce and/or minimize recovery time, reduce and/or minimize patient discomfort, reduce and/or minimize physician interventions, reduce and/or minimize a need and/or reliance on imaging or visualization to position the sponge, etc. For example, various aspects discussed herein allow for a porous and/or absorbent body, for example, a sponge, or treatment material to be delivered to a treatment site, for the sponge or treatment material to be removed (either degrade, physically removed, or a combination thereof). Additionally, various aspects discussed herein may be packaged as a kit to be used to treat a subject.

Although the aspects of the medical systems discussed above as being used to treat a treatment site in an esophagus, stomach, duodenum, large intestine (colon), or small intestine, these aspects and methods may be used to treat any portion of a subject, and may help to reduce the overall recovery time, component costs, reduce the risks to the subject, etc.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device, comprising:
a handle, wherein the handle includes a main body and a movable body, wherein the movable body is movable relative to the main body;
an end cap, wherein the end cap includes a stationary portion and an extendable portion, wherein the stationary portion includes one or more through-holes;
a sponge, wherein at least a portion of the sponge surrounds a portion of the extendable portion of the end cap;
one or more wires extending from the movable body of the handle, through the stationary portion of the end cap, and to the extendable portion of the end cap; and
one or more actuation elements connected to a portion of the extendable portion of the end cap,
wherein movement of the movable body of the handle controls a movement of the one or more wires and the extendable portion of the end cap, and
wherein proximal movement of the one or more actuation elements controls another movement of the extendable portion of the end cap to separate the extendable portion of the end cap from the sponge.

2. The medical device of claim 1, wherein the extendable portion of the end cap includes a band, and wherein the sponge is positioned radially around the band.

3. The medical device of claim 2, wherein the one or more wires are coupled to the band to extend and/or retract the band and the sponge.

4. The medical device of claim 1, wherein the main body includes a longitudinally extending slot, and wherein the movable body is moveable along at least a portion of the longitudinally extending slot.

5. The medical device of claim 1, further comprising a vacuum tube, wherein a distal end of the vacuum tube is coupled to a proximal portion of the sponge.

6. The medical device of claim 5, wherein the distal end of the vacuum tube is embedded within portion of the sponge.

7. The medical device of claim 5, wherein a proximal end of the vacuum tube is coupled to a vacuum or negative pressure source.

8. The medical device of claim 1, further comprising one or more tubes extending from the main body of the handle to the stationary portion of the end cap, wherein the one or more wires are each movable within a respective one of the one or more tubes.

9. The medical device of claim 8, wherein distal ends of the one or more tubes are fixed to the stationary portion of the end cap at the through-holes.

10. The medical device of claim 9, wherein the one or more through-holes includes two through-holes, wherein the one or more wires includes two wires, and wherein the one or more tubes includes two tubes.

11. The medical device of claim 10, wherein the two through-holes are evenly spaced around the stationary portion of the end cap.

12. The medical device of claim 1, wherein the sponge expands radially inward when separated from the extendable portion of the end cap.

13. The medical device of claim 1, wherein the handle includes a ring at a proximal end of the handle.

14. The medical device of claim 1, wherein the end cap is configured to be coupled to an exterior of a distal portion of an insertion shaft of an insertion device.

15. The medical device of claim 1, further comprising an actuator, wherein proximal ends of each of the one or more actuation elements are coupled to the actuator such that rotation of the actuator proximally retracts the one or more threads to retract the extendable portion and deploy the sponge.

16. A medical system, comprising:
an insertion device, wherein the insertion device includes an insertion shaft with a distal end;
a suction device, wherein the suction device includes a suction tube;
a medical device, wherein the medical device includes:
a handle, wherein the handle includes a main body and a movable body, wherein the movable body is movable relative to the main body;
an end cap positioned around a distal portion of the insertion shaft,
wherein the end cap includes an extendable portion;
a sponge, wherein at least a portion of the sponge surrounds a portion of the extendable portion of the end cap;
one or more wires extending from the movable body of the handle, through the one or more through-holes of the stationary portion of the end cap, and to the extendable portion of the end cap; and
one or more actuation elements connected to a portion of the extendable portion of the end cap,
wherein movement of the movable body of the handle controls a movement of the one or more wires and the extendable portion of the end cap, and
wherein proximal movement of the one or more actuation elements controls another movement of the extendable portion of the end cap to separate the extendable portion of the end cap from the sponge.

17. The medical system of claim 16, wherein the distal end of the insertion device includes a working channel opening, one or more illumination devices, and one or more visualization devices.

18. The medical system of claim 16, further comprising an actuator, wherein proximal ends of each of the one or more actuation elements are coupled to the actuator such that rotation of the actuator proximally retracts the one or more actuation elements to retract the extendable portion and deploy the sponge.

19. A method of applying suction to a target site, comprising:
coupling one or more portions of an insertion device, a medical device, and a suction device to form a medical system,
wherein the medical device includes a handle with a movable body, an end cap including a stationary portion and an extendable portion, and a porous body releasably positioned around a portion of the extendable portion, and wherein the suction device includes a vacuum tube coupled to the porous body;
delivering various portions of the medical system to the target site;
positioning and/or deploying the porous body; and
applying negative pressure or suction to the target site.

20. The method of claim 19, wherein positioning the porous body includes moving the extendable portion of the end cap by moving the movable body of the handle, and wherein deploying the porous body includes proximally retracting one or more actuation elements coupled to extendable portion of the end cap.
